(19) United States
(12) Patent Application Publication
(10) Pub. No.: US 2026/0011780 A1
(43) Pub. Date: Jan. 8, 2026

(54) ELECTROLYTE SOLUTION FOR RECHARGEABLE LITHIUM BATTERY AND RECHARGEABLE LITHIUM BATTERY INCLUDING THE SAME

(71) Applicant: SAMSUNG SDI CO., LTD., Yongin-si (KR)

(72) Inventors: JINAH SEO, Yongin-si (KR); SUNDAE KIM, Yongin-si (KR); Sujeong KOH, Yongin-si (KR); Erang CHO, Yongin-si (KR); Bomee KWON, Yongin-si (KR); Euisoo JEONG, Yongin-si (KR); JIN-HYEOK LIM, Yongin-si (KR); SANGHOON KIM, Yongin-si (KR)

(21) Appl. No.: 19/057,754

(22) Filed: Feb. 19, 2025

(30) Foreign Application Priority Data
Jul. 2, 2024 (KR) 10-2024-0087034

(51) Int. Cl. H01M 10/0567; C07D 239/62; H01M 4/525; H01M 10/052; H01M 10/0569

(52) U.S. Cl. CPC H01M 10/0567; C07D 239/62; H01M 4/525; H01M 10/052; H01M 10/0569; H01M 2300/004

(57) ABSTRACT

Provided is an electrolyte solution for a rechargeable lithium battery and a rechargeable lithium battery including the same, and the electrolyte solution includes a non-aqueous organic solvent, a lithium salt, and an additive represented by Formula 1 as described in the disclosure.

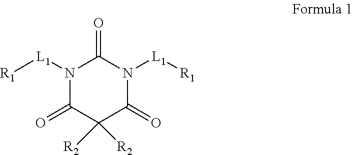

### CROSS-REFERENCE TO RELATED APPLICATION

The present application claims priority to and the benefit of Korean Patent Application No. 10-2024-0087034, filed on Jul. 2, 2024, in the Korean Intellectual Property Office, the entire disclosure of which is hereby incorporated by reference.

### BACKGROUND

### 1. Field

Embodiments of the present disclosure herein relate to an electrolyte solution for a rechargeable lithium battery, and a rechargeable lithium battery including the same.

### 2. Description of the Related Art

Recently, with the rapid spread of battery-using electronic devices, such as mobile phones, laptop computers, and electric vehicles, demand for a rechargeable battery having high energy density and high capacity has been rapidly increased. Accordingly, research and development has been actively conducted to improve performance of rechargeable lithium batteries.

Rechargeable lithium batteries may include a positive electrode and a negative electrode including an active material capable of intercalation and deintercalation of lithium ions, and an electrolyte solution. Electrical energy is produced by oxidation and reduction reactions if (e.g., when) the lithium ions are intercalated and deintercalated into/from the positive electrode and the negative electrode.

A non-aqueous organic solvent in which a lithium salt is dissolved may be used as an electrolyte for this rechargeable lithium battery. The rechargeable lithium battery may have battery characteristics through complex reactions between the positive electrode and the electrolyte, the negative electrode and the electrolyte, and/or the like. Therefore, the use of a suitable or appropriate electrolyte is one of the important variables that improve performance of rechargeable lithium batteries.

### SUMMARY

Embodiments of the present disclosure provide an electrolyte solution for a rechargeable lithium battery having improved lifespan characteristics and stability at high temperature.

Embodiments of the present disclosure also provide a rechargeable lithium battery including the electrolyte solution.

An embodiment of the present disclosure provides an electrolyte solution for a rechargeable lithium battery including a non-aqueous organic solvent, a lithium salt, and an additive represented by Formula 1.

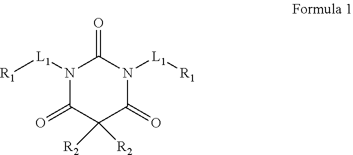

In Formula 1,

each L₁, independently, is a substituted or unsubstituted C1 to C20 alkylene group,

each R₁, independently, is a hydrogen atom, a substituted or unsubstituted C1 to C20 alkyl group, or a substituted or unsubstituted C2 to C20 alkenyl group, and at least one R₁ is a C2 to C20 alkenyl group, and

each R₂, independently, is a hydrogen atom, a substituted or unsubstituted C1 to C20 alkyl group, a substituted or unsubstituted C2 to C20 alkenyl group, or a substituted or unsubstituted C2 to C20 alkynyl group.

In an embodiment of the present disclosure, a rechargeable lithium battery includes a positive electrode including a positive electrode active material, a negative electrode including a negative electrode active material, and an electrolyte solution, and the electrolyte solution includes a non-aqueous organic solvent, a lithium salt, and the additive represented by Formula 1 described herein.

In an embodiment of the present disclosure, a compound represented by Formula 1-1 is provided.

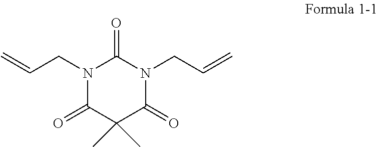 [SMILES: C=CCN1C(=O)N(CC=C)C(=O)C(C)(C)C1=O]

### BRIEF DESCRIPTION OF THE DRAWINGS

The accompanying drawings are included to provide a further understanding of embodiments of the present disclosure, and are incorporated in and constitute a part of this specification. The drawings illustrate embodiments of the present disclosure and, together with the description, serve to explain principles of the present disclosure. In the drawings:

FIG. 1 is a simplified conceptual diagram illustrating a rechargeable lithium battery according to embodiments of the present disclosure; and

FIGS. 2-5 are drawings schematically illustrating rechargeable lithium batteries according to embodiments.

### DETAILED DESCRIPTION

In order to fully understand the configuration and effect of the subject matter of the present disclosure, embodiments of the present disclosure will be described herein in more detail with reference to the accompanying drawings. The subject matter of the present disclosure may, however, be embodied in various suitable forms and should not be construed as being limited to the embodiments set forth herein, and various suitable changes and modifications can be made. Rather, these embodiments are provided so that this disclosure will be thorough and complete, and will fully convey the scope of the present disclosure to those having ordinary skill in the art to which the present disclosure pertains.

In this specification, it will be understood that, if (e.g., when) an element is referred to as being on another element, the element may be directly on the other element or intervening elements may be present therebetween. In the drawings, thicknesses of components may be exaggerated to effectively explain technical contents of the present disclosure. Like reference numerals or symbols refer to like elements throughout the specification.

The singular forms are intended to include the plural forms as well, unless the context clearly indicates otherwise. In embodiments, unless otherwise specially noted, the phrase “A or B” may indicate “including A but not B, B but not A, or A and B”. The terms “comprises/includes” and/or “comprising/including” used in this description do not exclude the presence or addition of one or more other components.

In this specification, “combination thereof” may refer to a mixture, a stack, a composite, a copolymer, an alloy, a blend, and/or a reaction product of components.

In this specification, unless defined otherwise, “substitution” means that at

least one hydrogen of a substituent or a compound is substituted with deuterium, a halogen group, a hydroxyl group, an amino group, a C1 to C30 amine group, a nitro group, a C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 fluoroalkyl group, a cyano group, or a combination thereof.

In some embodiments, “substitution” may mean that at least one hydrogen of a substituent or a compound is substituted with deuterium, a halogen group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C10 fluoroalkyl group, or a cyano group. For example, “substitution” may mean that at least one hydrogen of a substituent or a compound is substituted with deuterium, a halogen group, a C1 to C20 alkyl group, a C6 to C30 aryl group, a C1 to C10 fluoroalkyl group, or a cyano group. In some embodiments, “substitution” may mean that at least one hydrogen of a substituent or a compound is substituted with deuterium, a halogen group, a C1 to C5 alkyl group, a C6 to C18 aryl group, a C1 to C5 fluoroalkyl group, or a cyano group. For example, “substitution” may mean that at least one hydrogen of a substituent or a compound is substituted with deuterium, a cyano group, a halogen group, a methyl group, an ethyl group, a propyl group, a butyl group, a phenyl group, a biphenyl group, a terphenyl group, a trifluoromethyl group, or a naphthyl group.

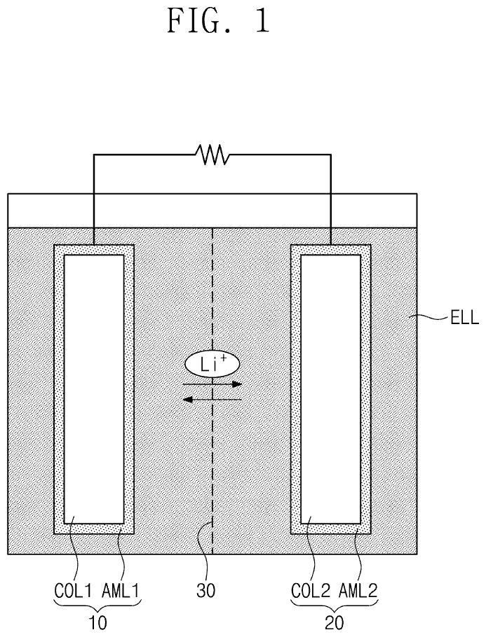

FIG. 1 is a cross-sectional view of a rechargeable lithium battery according to embodiments of the present disclosure. Referring to FIG. 1, the rechargeable lithium battery may include a positive electrode 10, a negative electrode 20, a separator 30, and an electrolyte solution ELL.

The positive electrode 10 and the negative electrode 20 may be spaced apart from each other by the separator 30. The separator 30 may be between the positive electrode 10 and the negative electrode 20. The positive electrode 10, the negative electrode 20 and the separator 30 may be in contact with the electrolyte solution ELL. The positive electrode 10, the negative electrode 20 and the separator 30 may be impregnated with the electrolyte solution ELL.

The electrolyte solution ELL may be a medium that transfers lithium ions between the positive electrode 10 and the negative electrode 20. In the electrolyte solution ELL, the lithium ions may move through the separator 30 toward the positive electrode 10 or the negative electrode 20.

### Positive Electrode 10

The positive electrode 10 for a rechargeable lithium battery may include a current collector COL1 and a positive electrode active material layer AML1 on the current collector. The positive electrode active material layer AML1 may include a positive electrode active material and may further include a binder and/or a conductive material (e.g., an electrically conductive material).

For example, the positive electrode 10 may further include an additive that can serve as a sacrificial positive electrode.

An amount of the positive electrode active material may be about 90 wt % to about 99.5 wt % based on 100 wt % of the positive electrode active material layer AML1. Amounts of the binder and the conductive material may be about 0.5 wt % to about 5 wt %, respectively, based on 100 wt % of the positive electrode active material layer AML1.

The binder serves to attach the positive electrode active material particles well to each other and also to attach the positive electrode active material well to the current collector COL1. Examples of the binder may include polyvinyl alcohol, carboxymethyl cellulose, hydroxypropyl cellulose, diacetyl cellulose, polyvinylchloride, carboxylated polyvinylchloride, polyvinylfluoride, a polymer including ethylene oxide, polyvinylpyrrolidone, polyurethane, polytetrafluoroethylene, polyvinylidene fluoride, polyethylene, polypropylene, a styrene-butadiene rubber, a (meth)acrylated styrene-butadiene rubber, an epoxy resin, a (meth)acrylic resin, a polyester resin, nylon, and the like, as non-limiting examples.

The conductive material may be used to impart conductivity (e.g., electrical conductivity) to the electrode. Any suitable material that does not cause chemical change (e.g., does not cause an undesirable chemical change in the rechargeable lithium battery) and conducts electrons can be used in the battery. Examples of the conductive material may include a carbon-based material such as natural graphite, artificial graphite, carbon black, acetylene black, ketjen black, a carbon fiber, a carbon nanofiber, and carbon nanotube; a metal-based material containing copper, nickel, aluminum, silver, etc., in a form of a metal powder and/or a metal fiber; a conductive polymer (e.g., an electrically conductive polymer) such as a polyphenylene derivative; or a mixture thereof.

Al may be used as the current collector COL1, but is not limited thereto.

### Positive Electrode Active Material

The positive electrode active material may include a compound (e.g., a lithiated intercalation compound) that is capable of intercalating and deintercalating lithium. For example, at least one of a composite oxide of lithium and a metal selected from cobalt, manganese, nickel, and combinations thereof may be used.

The composite oxide may be a lithium transition metal composite oxide. Examples of the composite oxide may include lithium nickel-based oxide, lithium cobalt-based oxide, lithium manganese-based oxide, lithium iron phosphate-based compound, cobalt-free nickel-manganese-based oxide, or a combination thereof.

As an example, any one of the following Chemical Formulas may be used. LiₐA_{1-b}X_{b}O_{2-c}D_{c} (0.90≤a≤1.8, 0≤b≤0.5, and 0≤c≤0.05); LiₐMn_{2-b}X_{b}O_{4-c}D_{c} (0.90≤a≤1.8, 0≤b≤0.5, and 0≤c≤0.05); LiₐNi_{1-b-c}Co_{b}X_{c}O_{2-α}D_{α} (0.90≤a≤1.8, 0≤b≤0.5, 0≤c≤0.5, and 0<α<2); LiₐNi_{1-b-c} Mn_{b}X_{c}O_{2-α}D_{α} (0.90≤a≤1.8, 0≤b≤0.5, 0≤c≤0.5, and 0<α<2); LiₐNi_{b}Co_{C}L¹_{d}GₑO₂ (0.90≤a≤1.8, 0≤b≤0.9, 0≤c≤0.5, 0≤d≤0.5, and 0≤e≤0.1); LiₐNiG_{b}O₂ (0.90≤a≤1.8 and 0.001≤b≤0.1); LiₐCoG_{b}O₂ (0.90≤a≤1.8 and 0.001≤b≤0.1); LiₐMn_{1-b}G_{b}O₂ (0.90≤a≤1.8 and 0.001≤b≤0.1); LiₐMn₂G_{b}O₄ (0.90≤a≤1.8 and 0.001≤b≤0.1); LiₐMn_{1-g}GgPO₄ (0.90≤a≤1.8 and 0≤g≤0.5); Li(_{3-f}) Fe₂(PO₄)₃ (0≤f≤2); or LiₐFePO₄ (0.90≤a≤1.8).

In the above Chemical Formulas, A is Ni, Co, Mn, or a combination thereof; X is Al, Ni, Co, Mn, Cr, Fe, Mg, Sr, V, a rare earth element or a combination thereof; D is O, F, S, P, or a combination thereof; G is Al, Cr, Mn, Fe, Mg, La, Ce, Sr, V, or a combination thereof; and L¹ is Mn, Al, or a combination thereof.

The positive electrode active material may be, for example, a high nickel-based positive electrode active material having a nickel content of greater than or equal to about 80 mol %, greater than or equal to about 85 mol %, greater than or equal to about 90 mol %, greater than or equal to about 91 mol %, or greater than or equal to about 94 mol % and less than or equal to about 99 mol % based on 100 mol % of the metal excluding lithium in the lithium transition metal composite oxide. The high-nickel-based positive electrode active material may be capable of realizing high capacity and can be applied to a high-capacity, high-density rechargeable lithium battery.

### Negative Electrode 20

The negative electrode 20 for a rechargeable lithium battery may include a current collector COL2 and a negative electrode active material layer AML2 on the current collector COL2. The negative electrode active material layer AML2 may include a negative electrode active material, and may further include a binder and/or a conductive material (e.g., an electrically conductive material).

For example, the negative electrode active material layer AML2 may include about 90 wt % to about 99 wt % of the negative electrode active material, about 0.5 wt % to about 5 wt % of the binder, and about 0 wt % to about 5 wt % of the conductive material.

The binder may serve to attach the negative electrode active material particles well to each other and also to attach the negative electrode active material well to the current collector COL2. The binder may include a non-aqueous binder, an aqueous binder, a dry binder, or a combination thereof.

The non-aqueous binder may include polyvinyl chloride, carboxylated

polyvinyl chloride, polyvinyl fluoride, an ethylene propylene copolymer, polystyrene, polyurethane, polytetrafluoroethylene, polyvinylidene fluoride, polyethylene, polypropylene, poly amideimide, polyimide, or a combination thereof.

The aqueous binder may be selected from a styrene-butadiene rubber, a (meth)acrylated styrene-butadiene rubber, a (meth)acrylonitrile-butadiene rubber, (meth)acrylic rubber, a butyl rubber, a fluoro rubber, polyethylene oxide, polyvinylpyrrolidone, polyepichlorohydrine, polyphosphazene, poly(meth)acrylonitrile, an ethylene propylene diene copolymer, polyvinylpyridine, chlorosulfonated polyethylene, latex, a polyester resin, a (meth)acrylic resin, a phenol resin, an epoxy resins, polyvinyl alcohol, and a combination thereof.

If (e.g., when) an aqueous binder is used as the negative electrode binder, a cellulose-based compound capable of imparting or increasing viscosity may be further included. The cellulose-based compound may include at least one of carboxymethyl cellulose, hydroxypropylmethyl cellulose, methyl cellulose, or an alkali metal salt thereof. The alkali metal may include Na, K, and/or Li.

The dry binder may be a polymer material that is capable of being fibrous (e.g., capable of being fiberized). For example, the dry binder may be polytetrafluoroethylene, polyvinylidene fluoride, a polyvinylidene fluoride-hexafluoropropylene copolymer, polyethylene oxide, or a combination thereof.

The conductive material may be used to impart conductivity (e.g., electrical conductivity) to the electrode. Any suitable material that does not cause chemical change (e.g., does not cause an undesirable chemical change in the rechargeable lithium battery) and that conducts electrons can be used in the battery. Non-limiting examples thereof may include a carbon-based material such as natural graphite, artificial graphite, carbon black, acetylene black, ketjen black, a carbon fiber, a carbon nanofiber, and a carbon nanotube; a metal-based material including copper, nickel, aluminum, silver, etc. in a form of a metal powder and/or a metal fiber; a conductive polymer such (e.g., an electrically conductive polymer) as a polyphenylene derivative; or a mixture thereof.

The negative current collector COL2 may include a copper foil, a nickel foil, a stainless steel foil, a titanium foil, a nickel foam, a copper foam, a polymer substrate coated with a conductive metal, or a combination thereof.

### Negative Electrode Active Material

The negative electrode active material may include a material that reversibly intercalates/deintercalates lithium ions, a lithium metal, a lithium metal alloy, a material capable of doping/dedoping lithium, and/or a transition metal oxide.

The material that reversibly intercalates/deintercalates lithium ions may include a carbon-based negative electrode active material, such as, for example, crystalline carbon, amorphous carbon or a combination thereof. The crystalline carbon may be graphite such as non-shaped, sheet-shaped, flake-shaped, sphere-shaped, and/or fiber-shaped natural graphite and/or artificial graphite. The amorphous carbon may be a soft carbon, a hard carbon, a mesophase pitch carbonization product, calcined coke, and/or the like.

The lithium metal alloy includes an alloy of lithium and a metal selected from Na, K, Rb, Cs, Fr, Be, Mg, Ca, Sr, Si, Sb, Pb, In, Zn, Ba, Ra, Ge, Al, and Sn.

The material capable of doping/dedoping lithium may be a Si-based negative electrode active material and/or a Sn-based negative electrode active material. The

Si-based negative electrode active material may include silicon, a silicon-carbon composite, SiOx (0≤x≤2), a Si-Q alloy (where Q is selected from an alkali metal, an alkaline-earth metal, a Group 13 element, a Group 14 element (excluding Si), a Group 15 element, a Group 16 element, a transition metal, a rare earth element, and a combination thereof). The Sn-based negative electrode active material may include Sn, SnO₂, a Sn-based alloy, or a combination thereof.

The silicon-carbon composite may be a composite of silicon and amorphous carbon. According to an embodiment, the silicon-carbon composite may be in a form of silicon particles and amorphous carbon coated on the surface of the silicon particles. For example, the silicon-carbon composite may include a secondary particle (core) in which primary silicon particles are assembled, and an amorphous carbon coating layer (shell) on the surface of the secondary particle. The amorphous carbon may also be between the primary silicon particles, and, for example, the primary silicon particles may be coated with the amorphous carbon. The secondary particle may exist dispersed in an amorphous carbon matrix.

The silicon-carbon composite may further include crystalline carbon. For example, the silicon-carbon composite may include a core including crystalline carbon and silicon particles and an amorphous carbon coating layer on a surface of the core.

The Si-based negative electrode active material and/or the Sn-based negative electrode active material may be used in combination with a carbon-based negative electrode active material.

### Separator 30

Depending on the type (or kind) of the rechargeable lithium battery, the separator 30 may be present between the positive electrode 10 and the negative electrode 20. The separator 30 may include polyethylene, polypropylene, polyvinylidene fluoride, or a multilayer film of two or more layers thereof, and a mixed multilayer film such as a polyethylene/polypropylene two-layer separator, polyethylene/polypropylene/polyethylene three-layer separator, polypropylene/polyethylene/polypropylene three-layer separator, and/or the like.

The separator 30 may include a porous substrate and a coating layer including an organic material, an inorganic material, or a combination thereof on one or both surfaces (e.g., two opposing surfaces) of the porous substrate.

The porous substrate may be a polymer film formed of any one selected polymer polyolefin such as polyethylene and polypropylene, polyester such as polyethylene terephthalate and polybutylene terephthalate, polyacetal, polyamide, polyimide, polycarbonate, polyether ketone, polyarylether ketone, polyether ketone, polyetherimide, polyamideimide, polybenzimidazole, polyethersulfone, polyphenylene oxide, a cyclic olefin copolymer, polyphenylene sulfide, polyethylene naphthalate, a glass fiber, TEFLON, and polytetrafluoroethylene, or a copolymer or mixture of two or more thereof.

The organic material may include a polyvinylidene fluoride-based polymer and/or a (meth)acrylic polymer.

The inorganic material may include inorganic particles selected from Al₂O₃, SiO₂, TiO₂, SnO₂, CeO₂, MgO, NiO, CaO, GaO, ZnO, ZrO₂, Y₂0₃, SrTiO₃, BaTiO_{3,} Mg(OH)₂, boehmite, and a combination thereof, but is not limited thereto.

The organic material and the inorganic material may be mixed in one coating layer, or a coating layer including an organic material and a coating layer including an inorganic material may be stacked.

### Electrolyte Solution ELL

The electrolyte solution ELL for a rechargeable lithium battery may include a non-aqueous organic solvent and a lithium salt.

The non-aqueous organic solvent may serve as a medium that transmits ions taking part in the electrochemical reaction of a battery.

The non-aqueous organic solvent may be a carbonate-based, ester-based, ether-based, ketone-based, and/or alcohol-based solvent, an aprotic solvent, or a combination thereof.

The carbonate-based solvent may include dimethyl carbonate (DMC), diethyl carbonate (DEC), dipropyl carbonate (DPC), methylpropyl carbonate (MPC), ethylpropyl carbonate (EPC), methylethyl carbonate (MEC), ethylene carbonate (EC), propylene carbonate (PC), butylene carbonate (BC), and/or the like.

The ester-based solvent may include methyl acetate, ethyl acetate, n-propyl acetate, dimethyl acetate, methyl propionate, ethyl propionate, decanolide, mevalonolactone, valerolactone, caprolactone, and/or the like.

The ether-based solvent may include dibutyl ether, tetraglyme, diglyme, dimethoxyethane, 2-methyltetrahydrofuran, 2,5-dimethyltetrahydrofuran, tetrahydrofuran, and/or the like. In some embodiments, the ketone-based solvent may include cyclohexanone, and/or the like. The alcohol-based solvent may include ethanol, isopropyl alcohol, and/or the like and the aprotic solvent may include nitriles such as R-CN (wherein R is a C2 to C20 linear, branched, or cyclic hydrocarbon group, a double bond, an aromatic ring, and/or an ether bond, and/or the like); amides such as dimethylformamide; dioxolanes such as 1,3-dioxolane, 1,4-dioxolane, and/or the like; sulfolanes, and/or the like.

The non-aqueous organic solvents may be used alone or in combination of two or more.

In some embodiments, if (e.g., when) using a carbonate-based solvent, a cyclic carbonate and a chain carbonate may be mixed and used, and the cyclic carbonate and the chain carbonate may be mixed in a volume ratio of about 1:1 to about 1:9.

The lithium salt dissolved in the organic solvent supplies lithium ions in a battery, enables a basic operation of a rechargeable lithium battery, and improves transportation of the lithium ions between positive and negative electrodes. Examples of the lithium salt include at least one selected from LiPF₆, LiBF₄, LiSbF₆, LiAsF₆, LiClO₄, LiAlO₂, LiAlCl₄, LiPO₂F₂, LiCl, LiI, LiN(SO₃C₂F₅)₂, Li (FSO₂)₂N (lithium bis(fluorosulfonyl)imide, LiFSI), LiC₄F₉SO₃, LiN(CₓF₂ₓ₊₁SO₂)(C_{y}F_{2y+1}SO₂) (wherein x and y are integers of 1 to 20), lithium trifluoromethane sulfonate, lithium tetrafluoroethanesulfonate, lithium difluoro(oxalato) borate (LiDFOB), lithium difluorobis(oxalato)phosphate (LiDFBOP), and lithium bis(oxalato) borate (LiBOB).

### Rechargeable Lithium Battery

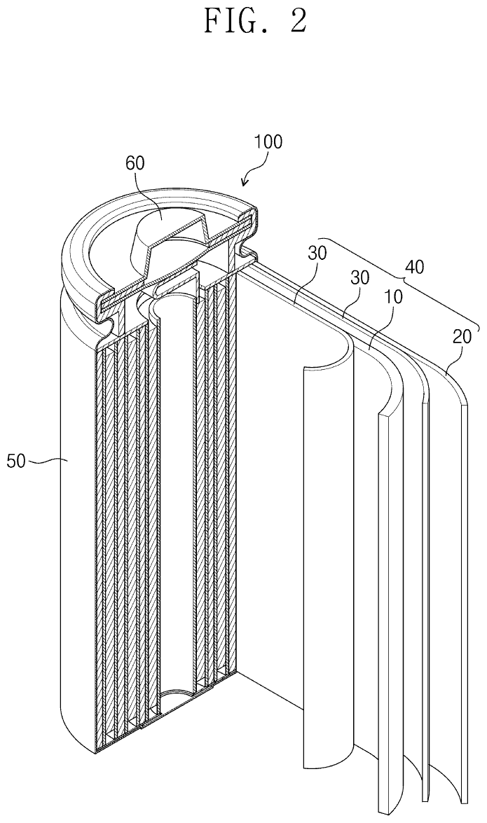

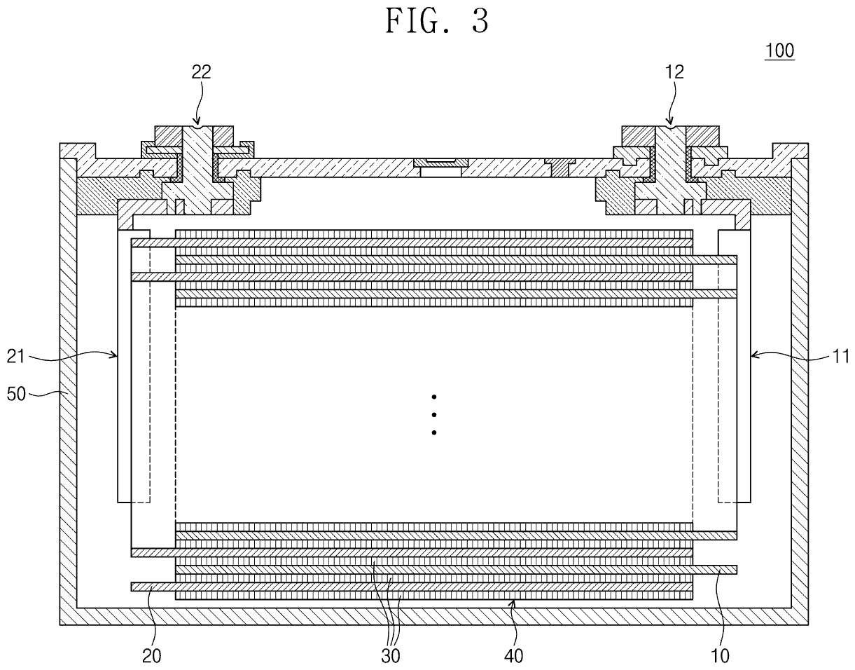

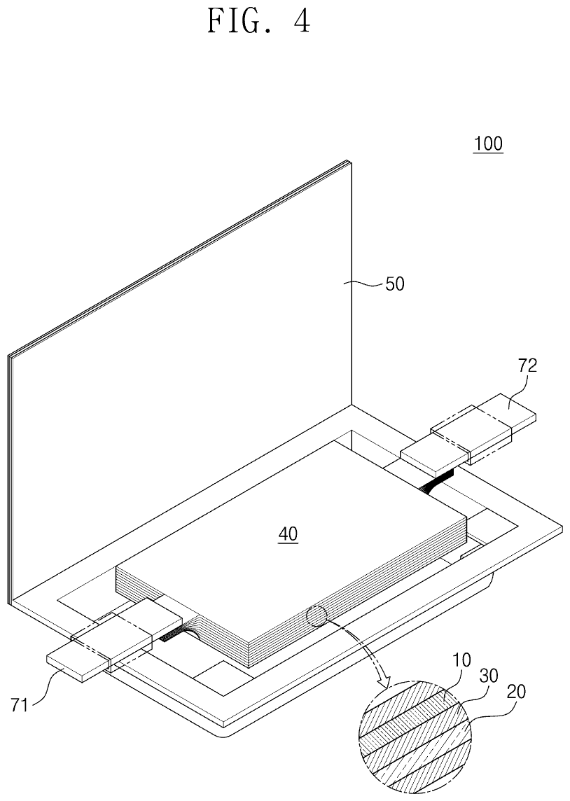

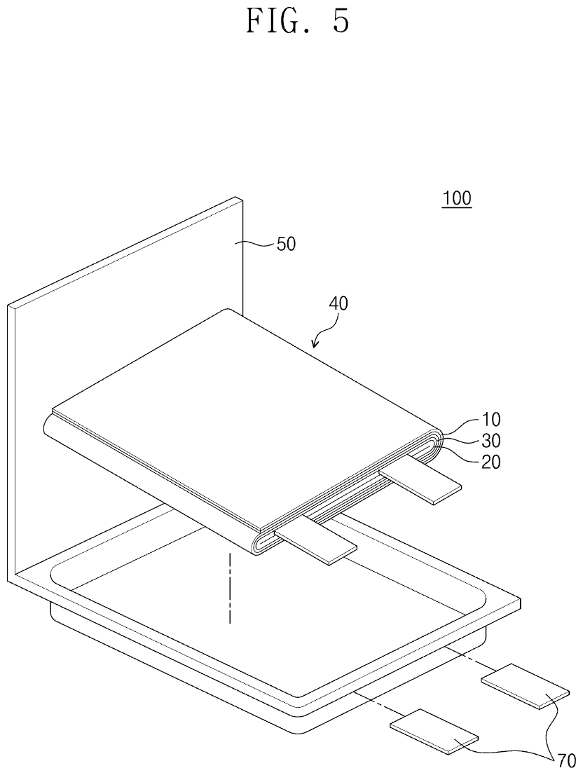

The rechargeable lithium battery may be classified into cylindrical, prismatic, pouch, or coin-type batteries, and/or the like depending on their shape. FIGS. 2-5 are schematic views illustrating rechargeable lithium batteries according to embodiments. FIG. 2 shows a cylindrical battery, FIG. 3 shows a prismatic battery, and FIGS. 4-5 show pouch-type batteries. Referring to FIGS. 2-5, the rechargeable lithium battery 100 may include an electrode assembly 40 including a separator 30 between a positive electrode 10 and a negative electrode 20, and a case 50 in which the electrode assembly 40 is included. The positive electrode 10, the negative electrode 20, and the separator 30 may be impregnated with an electrolyte solution. The rechargeable lithium battery 100 may include a sealing member 60 that seals the case 50, as shown in FIG. 2. In FIG. 3, the rechargeable lithium battery 100 may include a positive lead tab 11, a positive terminal 12, a negative lead tab 21, and a negative terminal 22. As shown in FIGS. 4-5, the rechargeable lithium battery 100 may include an electrode tab 70 (FIG. 5), which may be, for example, a positive electrode tab 71 and a negative electrode tab 72 (FIG. 4) that serve as an electrical path that induces the current formed in the electrode assembly 40 to the outside.

Hereinafter, an electrolyte solution for a rechargeable lithium battery according to embodiments of the present disclosure will be described in more detail.

The electrolyte solution for the rechargeable lithium battery, according to an embodiment, includes a non-aqueous organic solvent, a lithium salt, and an additive.

The additive according to an embodiment of the present disclosure may be represented by Formula 1:

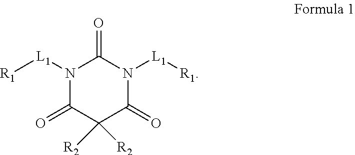

In Formula 1, each L₁, independently, may be a substituted or unsubstituted C1 to C20 alkylene group.

In Formula 1, each R₁, independently, may be a hydrogen atom, a substituted or unsubstituted C1 to C20 alkyl group, or a substituted or unsubstituted C2 to C20 alkenyl group, and at least one R₁ above may be a C2 to C20 alkenyl group.

In Formula 1, each R₂, independently, may be a hydrogen atom, a substituted or unsubstituted C1 to C20 alkyl group, a substituted or unsubstituted C2 to C20 alkenyl group, or a substituted or unsubstituted C2 to C20 alkynyl group.

For example, in Formula 1, one R₁ may include a double bond. For example, in Formula 1 above, all R₁ may include a double bond.

The additive represented by Formula 1 may be represented by Formula 1-1. In an embodiment, the additive of the electrolyte solution for the rechargeable lithium battery according to an embodiment of the present disclosure may be a compound represented by Formula 1-1:

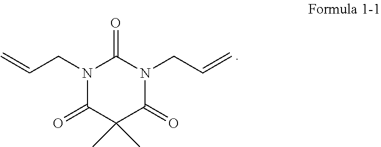 [SMILES: C=CCN1C(=O)N(CC=C)C(=O)C(C)(C)C1=O]

The additive according to an embodiment of the present disclosure has a structure of a cyclic compound including a nitrogen atom (N) substituted with a vinyl group. The nitrogen atom (N) present in the cyclic compound may combine with metal ions of a positive electrode active material to reduce surface deterioration. Accordingly, residual lithium in the positive electrode active material may be removed, and side reactions on the surface of the positive electrode active material may be suppressed or reduced, thereby suppressing or reducing lithium elution.

The additive according to an embodiment of the present disclosure may include at least one vinyl group. In some embodiments, because the additive, represented by Formula 1, has the vinyl group at a terminal (e.g., at a terminal end), polymerization may occur easily and consecutively if (e.g., when) a solid electrolyte interphase (SEI) film is formed. Accordingly, due to the consecutive polymerization, the additive according to an embodiment of the present disclosure forms a relatively thick and high-density SEI film, which is excellent in stability.

Because the additive according to an embodiment of the present disclosure has a structure of a cyclic compound including a nitrogen atom substituted with a vinyl group, it may be effective in improving lifespan characteristics and stability at a high-temperature condition if (e.g., when) a rechargeable battery is activated.

An effect of the additive, represented by Formula 1, on improving high-temperature stability of the rechargeable lithium battery is more noticeable if (e.g., when) used along with a high-nickel-based positive electrode active material and a negative electrode active material containing a silicon-carbon composite. In some embodiments, a silicon particle may be used for increasing a battery capacity, but may cause a side reaction with an electrolyte solution, so that resistance (e.g., electrical resistance) inside the battery may be increased. Because the side reaction of the silicon particle with the electrolyte solution is suppressed or reduced by the above-mentioned additive through stable formation of a film on a negative electrode, the purpose of increasing the battery capacity may be maximized or increased while the increase of the resistance (e.g., electrical resistance) inside the battery may be minimized or reduced.

The additive may be included in an amount (e.g., weight) of about 0.01 wt % to about 10 wt % on the basis of the total amount (e.g., weight) of the electrolyte solution. In some embodiments, the amount of the additive may be about 0.05 wt % or greater, about 0.1 wt % or greater, and about 0.5 wt % or greater on the basis of the total amount of the electrolyte solution. The amount of the additive may be about 8 wt % or less, about 7 wt % or less, and about 5 wt % or less on the basis of the total amount of the electrolyte solution.

If (e.g., when) the amount of the additive is below the above-mentioned ranges, the film may not be formed sufficiently on a lithium-based positive electrode and negative electrode, and if (e.g., when) the amount of the additive exceeds the above-mentioned ranges, the battery capacity and high-temperature lifespan may decrease, due to increase in resistance (e.g., electrical resistance) of the positive electrode and the negative electrode.

The electrolyte solution may be prepared through a mixed process in which a lithium salt is dissolved in a non-aqueous organic solvent, and the additive is added. A mixing process of the electrolyte solution, may be any suitable one generally used in the field of preparing the electrolyte solution, may be suitably or appropriately selected and used by those having ordinary skill in the art.

The non-aqueous organic solvent may include at least one selected from the group consisting of ethyl methyl carbonate (EMC), ethylene carbonate (EC), dimethyl carbonate (DMC), propylene carbonate (PC), propylpropionate (PP), diethyl carbonate (DEC), dipropyl carbonate (DPC), methylpropyl carbonate (MPC), ethylpropyl carbonate (EPC), methylethyl carbonate (MEC), and butylene carbonate (BC).

For example, the non-aqueous organic solvent may be a mixed solvent of ethyl methyl carbonate (EMC), ethylene carbonate (EC), and dimethyl carbonate (DMC).

For example, the ethyl methyl carbonate (EMC) may be included in an amount of about 10 vol % to about 40 vol % on the basis of the total amount of the non-aqueous organic solvent. The ethylene carbonate (EC) may be included in an amount of about 10 vol % to about 40 vol % on the basis of the total amount of the non-aqueous organic solvent. The dimethyl carbonate (DMC) may be included in an amount of about 30 vol % to about 80 vol % on the basis of the total amount of the non-aqueous organic solvent.

The lithium salt may include at least one selected from the group consisting of LiPF₆, LiBF₄, LiSbF₆, LiAsF₆, LiClO₄, LiAlO₂, LiAlCl₄, LiPO₂F2, LiCl, LiI, LiN(SO₃C₂F₅)₂, lithium bis(fluorosulfonyl)imide (Li(FSO₂)₂N, LiFSI), and LiC₄F₉SO₃. According to an embodiment, LiPF₆ may be used as the lithium salt.

The lithium salt may have a concentration of about 0.1 M to about 2.0 M. In some embodiments, the concentration of the lithium salt may be about 0.5 M or greater, and about 1.0 M or greater. The concentration of the lithium salt may be about 2.0 M or less, about 1.7 M or less, or about 1.5 M or less. According to an embodiment of the present disclosure, if (e.g., when) the concentration of the lithium salt is about 0.1 M to about 2.0 M, conductivity (e.g., electrical conductivity) and viscosity of the electrolyte solution may be suitably or appropriately maintained.

According to another embodiment of the present disclosure, a rechargeable lithium battery may include a positive electrode containing a positive electrode active material; a negative electrode including a negative electrode active material; and an electrolyte solution, and the electrolyte solution may include a non-aqueous organic solvent; a lithium salt; and the additive represented by Formula 1 described herein.

The positive electrode active material may include a lithium composite oxide represented by Formula 2.

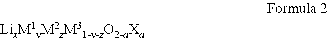

In Formula 2,

0.5≤x≤1.8, 0≤a≤0.05, 0<y≤1, 0≤z≤1, and 0≤y+z≤1 may be satisfied,

M¹, M², and M³ may each independently include at least one element selected from metal such as Ni, Co, Mn, Al, B, Ba, Ca, Ce, Cr, Fe, Mo, Nb, Si, Sr, Mg, Ti, V, W, Zr, or La, and a combination thereof.

X may include at least one element selected from F, S, P, or Cl.

In an embodiment, in Formula 2 above, M¹ may be Ni, and 0.8≤y≤1 and 0≤z≤0.2 may be satisfied.

In an embodiment, the positive electrode active material of the rechargeable lithium battery may include nickel, cobalt, and aluminum. In some embodiments, the positive electrode active material of the rechargeable lithium battery may include nickel, cobalt, and manganese. For example, in some embodiments, the positive electrode active material may be lithium nickel cobalt aluminum oxide (NCA), lithium nickel cobalt manganese oxide (NCM), or a combination thereof.

The negative electrode active material may include at least one of graphite or a Si composite.

If (e.g., when) the negative electrode active material includes a Si composite and graphite together, the Si composite and the graphite may be included in the form of a mixture, and in this case, the Si composite and the graphite may be included in a weight ratio of about 1:99 to about 50:50. For example, the Si composite and the graphite may be included in a weight ratio of about 3:97 to about 20:80, or about 5:95 to about 20:80.

The Si composite may include a core containing a Si-based particle, and an amorphous carbon coating layer, and, for example, the Si-based particle may include at least one of a Si—C composite, SiOₓ (0<x≤ 2), or a Si alloy. For example, the Si—C composite may include a core containing a Si particle and crystalline carbon, and an amorphous carbon coating layer on a surface of the core.

The crystalline carbon may include, for example, graphite, and more particularly, natural graphite, artificial graphite, or a mixture thereof.

The rechargeable lithium battery may be applied to automobiles, mobile phones, and/or various suitable types (or kinds) of electric devices, and an embodiment of the present disclosure is not limited thereto.

Hereinafter, examples and comparative examples of the present disclosure are described. However, the following examples are provided for illustrative purpose only and are not to be construed to limit the scope of the present disclosure.

### EXAMPLE AND COMPARATIVE EXAMPLE

### Synthesis Example 1

10.0 g of diallyl urea was added in a round bottom flask, and 50 g of dichloromethane was added thereto. The mixture was agitated at room temperature (about 25° C.) until diallyl urea was dissolved, and then 13.3 g of 2,2-dimethylpropanedioyl dichloride was dissolved in 20 g of dichloromethane to be slowly added dropwise at room temperature. The reaction temperature was raised to about 40° C., the mixture was agitated for about 7 hours, and then the reaction was terminated. Thereafter, the reaction product was washed with distilled water (DIW) and then purified using column chromatography to obtain a compound represented by Formula 1-1.

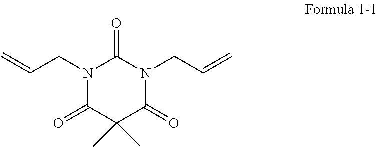 [SMILES: C=CCN1C(=O)N(CC=C)C(=O)C(C)(C)C1=O]

* 1H NMR (400 MHZ, CDCl₃): 5.86-5.77 (m, 2H), 5.20 (dd, J=17.0, 10.1 Hz, 4H), 4.45 (d, J=4.5 Hz, 4H), 1.52 (s, 6H)

### Example 1

### (1) Preparation of Electrolyte Solution

1.0 M of LiPF₆ was dissolved in a non-aqueous organic solvent in which ethylene carbonate (EC), ethyl methyl carbonate (EMC), and dimethyl carbonate (DMC) were mixed in a volume ratio of about 20:20:60, and 0.1 wt % of an additive and 1.0 wt % of an auxiliary additive were added to prepare an electrolyte solution. Vinylene carbonate (VC) was used as the auxiliary additive.

The compound represented by Formula 1-1, prepared according to Synthesis Example 1, was used for the additive.

### (2) Preparation of Rechargeable Lithium Battery

97 wt % of NCA (LiNi_{0.91}Co_{0.08}Al_{0.01}O₂) as a positive electrode active material, and as a conductive material, 0.5 wt % of artificial graphite powder, 0.8 wt % of carbon black (ketjen black), 0.2 wt % of acrylonitrile rubber, and 1.5 wt % of polyvinylidene fluoride (PVdF) were mixed and added to N-methyl-2-pyrrolidone, and then agitated for about 30 minutes using a mechanical agitator to prepare a positive electrode active material slurry. A doctor blade was used to apply the positive electrode active material slurry to a thickness of about 60 μm onto an aluminum current collector having a thickness of about 20 μm, the applied positive electrode active material slurry was dried in a hot-air drier at about 100° C. for about 0.5 hours, then dried again for about 4 hours at about 120° C. in a vacuum condition, and roll-pressed to prepare a positive electrode.

98 wt % of a negative electrode active material in which artificial graphite and a Si composite were mixed in a weight ratio of about 93:7, 1 wt % of styrene-butadiene rubber (SBR), and 1 wt % of carboxymethyl cellulose (CMC) were mixed and then added to distilled water, and agitated for about 60 minutes using a mechanical agitator to prepare a negative electrode active material slurry. A doctor blade was used to apply the negative electrode active material slurry to a thickness of about 60 μm onto a copper current collector having a thickness of about 10 μm, the applied negative electrode active material slurry was dried in a hot-air drier at about 100° C. for about 0.5 hours, then dried again for about 4 hours at about 120° C. in a vacuum condition, and roll-pressed to prepare a negative electrode.

The positive electrode, the negative electrode, and a polyethylene separator having a thickness of about 10 μm were assembled to prepare an electrode assembly, and the electrolyte solution was introduced to prepare a rechargeable lithium battery.

### Example 2

An electrolyte solution and a rechargeable lithium battery were prepared in substantially the same manner as that of Example 1 except that 0.5 wt % of an additive was applied.

### Example 3

An electrolyte solution and a rechargeable lithium battery were prepared in substantially the same manner as that of Example 1 except that 2.0 wt % of an additive was applied.

### Example 4

An electrolyte solution and a rechargeable lithium battery were prepared in substantially the same manner as that of Example 1 except that 5.0 wt % of an additive was applied.

### Comparative Example 1

An electrolyte solution and a rechargeable lithium battery were prepared in substantially the same manner as that of Example 1 except that the additive represented by Formula 1-1 was not added in the preparation of the electrolyte solution.

### Evaluation Example

A rechargeable lithium battery was evaluated in the following methods.

### Evaluation 1: Evaluation on High-Temperature Storage Characteristics

A value of ΔV/ΔI (voltage change/current change) for initial direct current internal resistance (DC-IR) of each of the rechargeable lithium batteries, prepared according to examples and a comparative example, was measured, then the maximum energy state inside the battery was made to a full-charge state (SOC 100%), and in this state, the battery was stored at high temperature (about 60° C.) for 30 days, and then DC-IR was measured. The DC-IR increase rate was calculated according to Equation 1 below, and the results are listed in Table 1.

Initial discharge capacities of the rechargeable lithium batteries, prepared according to examples and a comparative example, and the discharge capacities thereof after storage at high temperature (about 60° C.) for 30 days were each measured. The capacity retention rate was calculated according to Equation 2 below, and the results are listed in Table 1.

$$DC - IR increase rate = ( DC - IR after 30 days / initial DC - IR ) \times 100 \qquad Equation 1$$

$$Capacity retention rate ( \% ) = ( discharge capacity after 30 days / initial discharge capacity ) \times 100 \qquad Equation 2$$

TABLE 1

| | DC-IR increase | Capacity retention rate |
|---|---|---|
| Classification | rate (%) | [%] |
| Comparative Example | 151 | 83 |
| 1 | | |
| Example 1 | 149 | 86 |
| Example 2 | 135 | 92 |
| Example 3 | 128 | 94 |
| Example 4 | 126 | 95 |

### Evaluation 2: Evaluation on Characteristics of Gas Generation at High Temperature

Characteristics of high-temperature gas generation of the rechargeable lithium batteries, according to examples and a comparative example, were evaluated.

For this, the rechargeable lithium batteries, according to the examples and comparative example, were charged at about 45° C. and about 4.2 V, and then left to stand at about 60° C. for 30 days.

In order to observe gas reduction effect, the amount of initial gas generation of the rechargeable lithium batteries, prepared according to the examples and comparative example, and the amount of gas generation of the batteries left to stand for 30 days were measured. The amount of gas generation was calculated according to Equation 3 below, and the results were listed in Table 2 below.

$$Amount of gas generation ( \% ) = ( amount of gas generation after 30 days / amount of initial gas generation ) \times 100 \qquad Equation 3$$

TABLE 2

| | Amount of gas generation when |
|---|---|
| | stored at high temperature of |
| | 60° C. (%) |
| Classification | 30 days |
| Comparative Example 1 | 62 |
| Example 1 | 51 |
| Example 2 | 45 |
| Example 3 | 23 |
| Example 4 | 19 |

### Evaluation 3: Evaluation on Lifespan Characteristics at High Temperature

Initial DC-IR values and initial discharge capacities of the rechargeable lithium batteries, prepared according to examples and a comparative example, were measured. Thereafter, under conditions of 0.33 C charging (CC/CV, 4.2 V cut-off)/1.0 C discharging (CC, 3.0 V cut-off) at high temperature (about 45° C.), a cycle of charging and discharging was performed 300 times on the rechargeable lithium batteries prepared according to the examples and comparative example, and then the DC-IR and discharge capacities were measured. The DC-IR increase rate and the capacity retention rate were calculated, and the results are listed in Table 3.

$$DC - IR increase rate = ( DC - IR after 300 cycles / initial DC - IR ) \times 100 \qquad Equation 4$$

$$Capacity retention rate ( \% ) = ( discharge capacity after 300 cycles / initial discharge capacity ) \times 100 \qquad Equation 5$$

TABLE 3

| | | Capacity retention rate at |
|---|---|---|
| | DC-IR increase rate | high temperature of 45° C. |
| Classification | (%) | (%) |
| Comparative | 144 | 76 |
| Example 1 | | |
| Example 1 | 140 | 78 |
| Example 2 | 135 | 82 |
| Example 3 | 128 | 83 |
| Example 4 | 133 | 81 |

### Comprehensive Evaluation

Referring to Table 1, it can be seen that, in case of using the electrolyte solution containing the additive according to embodiments of the present disclosure (Examples 1 to 4), storage characteristics of the batteries at high temperature (about 60° C.) were improved, compared to the case according to the comparative example.

Referring to Table 2, it can be seen that the rechargeable lithium battery prepared according to the comparative example had a relatively large amount of gas generation when stored at high temperature (about 60° C.), compared to the rechargeable lithium batteries prepared according to the examples. Therefore, in the rechargeable lithium battery where the additive according to embodiments of the present disclosure was used, gas generation at high temperature (about 60° C.) may be effectively suppressed.

Referring to Table 3, it can be seen that, in case of using the electrolyte solution containing the additive according to embodiments of the present disclosure (Examples 1 to 4), cycle characteristics and lifespan efficiency of the batteries, when stored at high temperature, were improved, compared to the case according to the comparative example.

An electrolyte solution for a rechargeable lithium battery according to an embodiment may have the effect of improving high-temperature lifespan characteristics and stability.

Although embodiments of the present disclosure have been described with reference to the accompanying drawings, it is understood that the present disclosure should not be limited to these embodiments, but various suitable changes and modifications can be made within the scope of the appended claims and equivalents thereof, the detailed description of the present disclosure, and the accompanying drawings, and this also falls within the scope of the present disclosure.

What is claimed is:

1. An electrolyte solution for a rechargeable lithium battery, comprising:
a non-aqueous organic solvent;
a lithium salt; and
an additive represented by Formula 1: 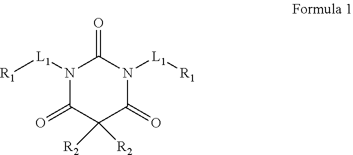
wherein, in Formula 1,
each L₁, independently, is a substituted or unsubstituted C1 to C20 alkylene group,
each R₁, independently, is a hydrogen atom, a substituted or unsubstituted C1 to C20 alkyl group, or a substituted or unsubstituted C2 to C20 alkenyl group, at least one R₁ being a C2 to C20 alkenyl group, and
each R₂, independently, is a hydrogen atom, a substituted or unsubstituted C1 to C20 alkyl group, a substituted or unsubstituted C2 to C20 alkenyl group, or a substituted or unsubstituted C2 to C20 alkynyl group.

2. The electrolyte solution as claimed in claim 1, wherein the additive is represented by Formula 1-1:

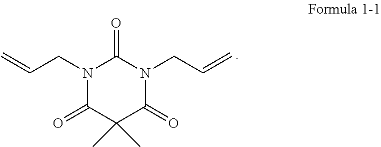 [SMILES: C=CCN1C(=O)N(CC=C)C(=O)C(C)(C)C1=O]

3. The electrolyte solution as claimed in claim 1, wherein the additive is included in an amount of about 0.01 wt % to about 10 wt % on the basis of a total weight of the electrolyte solution.

4. The electrolyte solution as claimed in claim 1, wherein the non-aqueous organic solvent comprises one or more selected from the group consisting of ethyl methyl carbonate (EMC), ethylene carbonate (EC), dimethyl carbonate (DMC), propylene carbonate (PC), propylpropionate (PP), diethyl carbonate (DEC), dipropyl carbonate (DPC), methylpropyl carbonate (MPC), ethylpropyl carbonate (EPC), methylethyl carbonate (MEC), and butylene carbonate (BC).

5. The electrolyte solution as claimed in claim 1, wherein the lithium salt is one or more selected from the group consisting of LiPF₆, LiBF₄, LiSbF₆, LiAsF₆, LiClO₄, LiAlO₂, LiAlCl₄, LiPO₂F₂, LiCl, LiI, LiN(SO₃C₂F₅)₂, lithium bis(fluorosulfonyl)imide (Li(FSO₂)₂N, LiFSI), and LiC₄F₉SO_{3.}

6. The electrolyte solution of claim 1, wherein the lithium salt is included in a concentration of about 0.1 M to about 2.0 M.

7. A rechargeable lithium battery comprising:
a positive electrode comprising a positive electrode active material;
a negative electrode comprising a negative electrode active material; and
the electrolyte solution as claimed in claim 1.

8. The rechargeable lithium battery as claimed in claim 7, wherein the positive electrode active material comprises a lithium composite oxide represented by Formula 2: 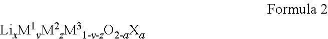
wherein, in Formula 2,
0.5≤x≤1.8, 0≤a≤0.05, 0<y≤1, 0≤z≤1, and 0≤y+z≤1,
M¹, M², and M³ each independently comprises one or more elements selected from metal comprising Ni, Co, Mn, Al, B, Ba, Ca, Ce, Cr, Fe, Mo, Nb, Si, Sr, Mg, Ti, V, W, Zr, La, or a combination thereof, and
X comprises one or more elements selected from F, S, P, or Cl.

9. The rechargeable lithium battery as claimed in claim 7, wherein the positive electrode active material comprises lithium nickel cobalt aluminum oxide (NCA), lithium nickel cobalt manganese oxide (NCM), or a combination thereof.

10. The rechargeable lithium battery as claimed in claim 7, wherein the negative electrode active material comprises at least one selected from among graphite and a Si composite.

11. The rechargeable lithium battery as claimed in claim 7, wherein the rechargeable lithium battery is a cylindrical, prismatic, pouch-type, or coin-type battery.

12. A compound represented by Formula 1-1 below: 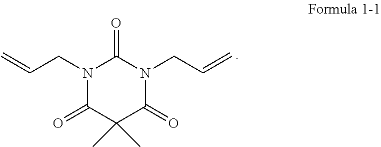 [SMILES: C=CCN1C(=O)N(CC=C)C(=O)C(C)(C)C1=O]
